(19) United States
(12) Patent Application Publication
(10) Pub. No.: US 2026/0000271 A1
(43) Pub. Date: Jan. 1, 2026

(54) SYSTEM TO ASSESS THE PRESENCE OF WATER AND/OR FOREIGN MATTER IN LUMINAL MEDICAL DEVICES

(71) Applicant: Boston Scientific Scimed, Inc., Maple Grove, MN (US)

(72) Inventor: Scott Edward Corbeil, Litchfield, NH (US)

(73) Assignee: Boston Scientific Scimed, Inc., Maple Grove, MN (US)

(21) Appl. No.: 19/249,198

(22) Filed: Jun. 25, 2025

Related U.S. Application Data
(60) Provisional application No. 63/664,373, filed on Jun. 26, 2024.

(51) Int. Cl. A61B 1/00; A61B 90/00; G01N 27/12; G08B 21/18; G08B 21/20

(52) U.S. Cl. CPC A61B 1/00057; A61B 90/06; G01N 27/121; G08B 21/182; G08B 21/20; G01N 27/122

(57) ABSTRACT

Methods and systems for detecting the presence of moisture and/or foreign matter in a lumen of a medical device. An illustrative method may comprise coupling a first probe to a first end of a lumen of a medical device, the first probe comprising a first electrode and a first electrical connection extending proximally from the first electrode, coupling a second probe to a second end of the lumen of the medical device, the second probe comprising a second electrode and a second electrical connection extending proximally from the second electrode, transmitting an electrical signal through the first and second electrical connections, determining a direct current (DC) voltage drop between the first and second electrical connections, calculating a surface resistivity of the lumen based on the DC voltage drop, determining if the calculated surface resistivity is below a threshold surface resistivity, and providing a test status notification.

### CROSS-REFERENCE TO RELATED APPLICATIONS

This application claims the benefit of U.S. Provisional Patent Application Ser. No. 63/664,373 filed on Jun. 26, 2024, the disclosure of which is incorporated herein by reference.

### FIELD

This disclosure relates generally to reusable luminal medical devices, and particularly to a methods and systems to determine a presence of moisture and/or foreign matter in a luminal medical device.

### BACKGROUND

Conventionally, endoscope devices have been widely used for performing diagnostic and/or therapeutic treatments. After use, endoscopes are required to go through a thorough cleaning and disinfection process. The cleaning and disinfection process may include many steps and at the end of the process, the external and internal surfaces are rinsed and/or flushed with water, rinsed and/or flushed with alcohol, dried, and properly stored in preparation for future use. Other reusable medical devices may be similarly processed for reuse. One of the challenges associated with endoscope reprocessing (or other medical device reprocessing) is ensuring that the devices are completely dry and/or free from foreign matter prior to storage and/or reuse. This may include the ensuring the internal lumens of the endoscope are dry. Inadequate cleaning, rinsing, and/or drying may impart risks to the reprocessing and reuse of devices. If there are any concerns about the lumens of a medical device being dry and/or free from foreign matter or if a facility mandates periodic quality control testing of the lumens to ensure they are dry and/or free from foreign matter, the devices may be inspected to check the lumen for moisture and/or foreign matter. For example, a borescope may be inserted into the lumen to visually inspect the lumen for moisture and/or foreign matter. However, once a borescope has been inserted into the lumen, the medical device is considered dirty and must be reprocessed and/or cleaned regardless of whether or not moisture or foreign matter was detected. This test and additional cleaning cycle are time consuming, increase facility labor costs, and only allows for random sampling for moisture and/or foreign matter.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a method for detecting the presence of moisture and/or foreign matter in a lumen of a medical device may comprise coupling a first probe to a first end of a lumen of a medical device, the first probe comprising a first electrode and a first electrical connection extending proximally from the first electrode, coupling a second probe to a second end of the lumen of the medical device, the second probe comprising a second electrode and a second electrical connection extending proximally from the second electrode, transmitting an electrical signal through the first electrical connection and the second electrical connection, determining a direct current (DC) voltage drop between the first electrical connection and the second electrical connection, calculating a surface resistivity of the lumen based on the DC voltage drop, determining if the calculated surface resistivity is below a threshold surface resistivity, and providing a test status notification.

Alternatively or additionally to any of the examples above, in another example, the method for detecting the presence of moisture and/or foreign matter in a lumen of a medical device may further comprise inputting a device type or model into a control and power element.

Alternatively or additionally to any of the examples above, in another example, the control and power element may be configured to use characteristics of the device type or model to calculate the surface resistivity of the lumen.

Alternatively or additionally to any of the examples above, in another example, the characteristics of the device type or model may comprise an inner diameter of the lumen and a distance between the first electrode and the second electrode.

Alternatively or additionally to any of the examples above, in another example, coupling the first probe to a first end of the lumen of the medical device may comprise positioning the first electrode in contact with an inner surface of the lumen.

Alternatively or additionally to any of the examples above, in another example, coupling the second probe to a second end of the lumen of the medical device may comprise positioning the second electrode in contact with an inner surface of the lumen.

Alternatively or additionally to any of the examples above, in another example, providing a test status notification may comprise displaying a numerical representation of the calculated surface resistivity.

Alternatively or additionally to any of the examples above, in another example, providing a test status notification may comprise displaying a binary representation of the presence of moisture and/or foreign matter in the lumen.

Alternatively or additionally to any of the examples above, in another example, providing a test status notification may comprise providing a test pass notification indicating the lumen is free from moisture and/or foreign matter or a test failure notification indicating moisture and/or foreign matter is present in the lumen.

In another example, a method for detecting the presence of moisture and/or foreign matter in a lumen of a medical device may comprise receiving at a power and control element a device type and/or model of a medical device, positioning a first electrode in contact with a first end of a lumen of the medical device, the first electrode in electrical communication with the power and control element, positioning a second electrode in contact with a second end of the lumen of the medical device, the second electrode in electrical communication with the power and control element, determining a direct current (DC) voltage drop between the first electrode and the second electrode, calculating a surface resistivity of the lumen based at least in part on the DC voltage drop, and determining if the calculated surface resistivity is below a threshold surface resistivity. If the calculated surface resistivity is below the threshold surface resistivity, the method may further comprise displaying a test failure notification, and if the calculated surface resistivity is not below the threshold surface resistivity the method may further comprise displaying a test pass notification.

Alternatively or additionally to any of the examples above, in another example, receiving at the power and control element the device type and/or model of the medical device may comprise receiving a user input at the power and control element.

Alternatively or additionally to any of the examples above, in another example, receiving at the power and control element the device type and/or model of the medical device may comprise scanning a machine-readable representation of information.

Alternatively or additionally to any of the examples above, in another example, the test failure notification and/or the test pass notification may comprise a visual alert.

Alternatively or additionally to any of the examples above, in another example, the test failure notification and/or the test pass notification may comprise an audio alert.

In another example, a system for detecting moisture and/or foreign matter in a lumen of a medical device may comprise a first probe comprising a first electrode and a first electrical connection extending proximally from the first electrode, a second probe comprising a second electrode and a second electrical connection extending proximally from the second electrode, and a power and control element including a processing unit and a power source. The power and control element may be electrically coupled to a proximal end of the first electrical connection and a proximal end of the second electrical connection. When the first electrode is electrically coupled to an inner surface of a lumen and the second electrode is electrically coupled to the inner surface of the lumen and spaced a distance from the first electrode, the power and control element may be configured to calculate a surface resistivity of the lumen.

Alternatively or additionally to any of the examples above, in another example, the first electrode may be coupled to an actuation member.

Alternatively or additionally to any of the examples above, in another example, actuation of the actuation member may be configured to radially displace the first electrode.

Alternatively or additionally to any of the examples above, in another example, the system for detecting moisture and/or foreign matter in a lumen of a medical device may further comprise a biasing member coupled to the first electrode and the actuation member.

Alternatively or additionally to any of the examples above, in another example, the first electrode may comprise a body portion having a tapered outer diameter.

Alternatively or additionally to any of the examples above, in another example, the power and control element may further comprise a display. The display may be configured to provide a positive or negative indication of the presence of moisture and/or foreign matter in the lumen.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 is a schematic diagram of a basic system for measuring surface resistivity of a generic object having a width;

FIG. 4 is a perspective view of an illustrative system for measuring surface resistivity in a luminal device;

FIG. 5A is a perspective view of a distal end region of an illustrative probe in a first configuration;

FIG. 5B is a perspective view of the distal end region of the illustrative probe of FIG. 5A in a second configuration;

FIG. 6 is a perspective view of a distal end region of another illustrative probe; and

FIG. 7 is a schematic flow chart of an illustrative method for using the system of FIG. 4 for measuring a surface resistivity of a device to detect moisture and/or foreign matter within a lumen of the device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular medical system and medical procedure is provided only for convenience and not intended to limit the disclosure. A person of ordinary skill in the art would recognize that the concepts underlying the disclosed devices and related methods of use may be utilized in any suitable device or procedure, medical or otherwise. This disclosure may be understood with reference to the following description and the appended drawings.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. The term “exemplary” is used in the sense of “example,” rather than “ideal.” Additionally, terms that indicate the geometric shape of a component/surface refer to exact and approximate shapes.

Although the present disclosure includes descriptions of an endoscope suitable for use with an endoscope system to assess the lumens thereof for moisture and/or foreign matter, the devices, systems, and methods herein could be implemented in other medical systems or devices having lumens, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. After use, endoscopes are required to go through a thorough cleaning and disinfection process. The cleaning and disinfection process may include many steps and at the end of the process, the external and internal surfaces are rinsed and/or flushed with water, rinsed, and/or flushed with alcohol, dried, and properly stored in preparation for future use. Other reusable medical devices may be similarly processed for reuse. One of the challenges associated with endoscope reprocessing (or other medical device reprocessing) is ensuring that the devices are completely dry and/or free from foreign matter prior to storage and/or reuse. This may include the internal lumens of the endoscope. Inadequate cleaning, rinsing, and/or drying may impart risks to the reprocessing and reuse of devices. If there are any concerns about the lumens of a medical device being dry or including a foreign matter or if a facility mandates periodic quality control testing of the lumens to ensure they are dry and/or free from a foreign matter, the devices may be inspected to check the lumen for moisture and/or foreign matter. For example, a borescope may be inserted into the lumen to visually inspect the lumen for moisture and/or foreign matter. However, once a borescope has been inserted into the lumen, the medical device is considered dirty and must be reprocessed and/or cleaned regardless of whether or not moisture and/or foreign matter was detected. This test and additional cleaning cycle are time consuming, increase facility labor costs, and only allows for random sampling for moisture and/or foreign matter. Disclosed herein are methods and systems for assessing the lumens of devices for moisture and/or foreign matter and/or water at the end of a reprocessing cycle.

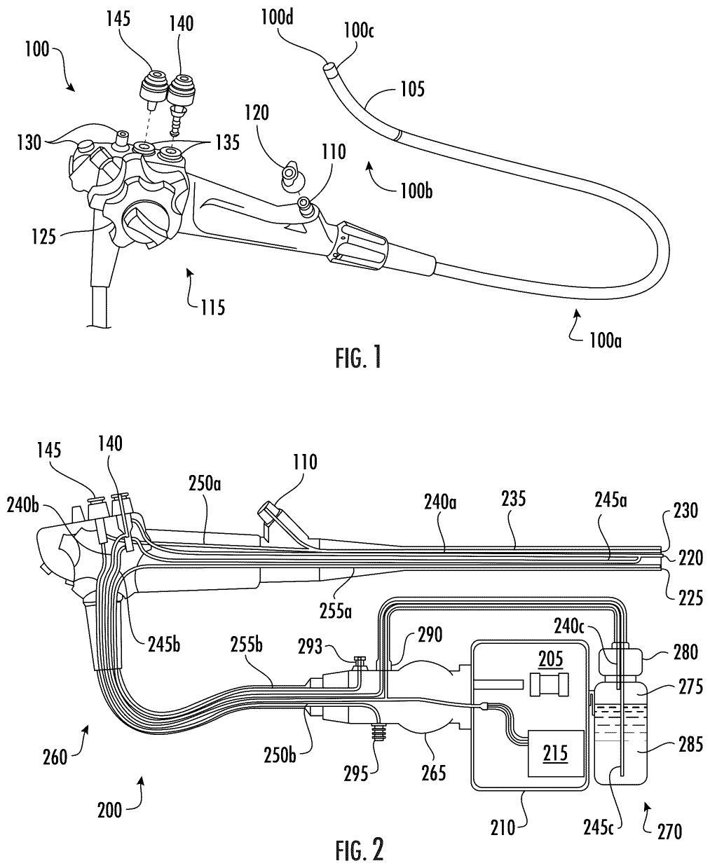

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid via an irrigation supply line 255a to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

After use, an endoscope 100 may be cleaned and disinfected so that the endoscope 100 may be reused in another procedure and/or on another patient. Specific details regarding the cleaning and disinfection procedure may be outlined in the device's instructions for use (IFU) documentation. Immediately after an endoscope 100 is used, the endoscope 100 may be treated to remove organic matters or materials from the device before said organic materials have an opportunity to dry. This cleaning step may occur in the procedure room and may include wiping the endoscope 100 with or without a cleaning agent or detergent. Next, the endoscope 100 may be transported to a reprocessing area where leak testing may occur, if required. It is contemplated that leak testing may detect potential damage to the surface and/or internal channels (e.g., working channel 235, gas supply line 240a, lens wash supply line 245a, suction supply line 250a, irrigation supply line 255a, gas feed line 240b, lens wash feed line 245b, suction feed line 250b, irrigation feed line 255b, or the like). Leak testing may be performed manually or may be automated. In some cases, guidelines or procedures for leak testing may be defined in the device's instructions for use documentation. If a leak and/or damage is identified, the endoscope 100 may be sent for repair or disposed of.

After the leak test has been completed, a manual cleaning of the endoscope 100 may occur. Manual cleaning may include, but is not limited to, brushing and flushing channels (e.g., working channel 235, gas supply line 240a, lens wash supply line 245a, suction supply line 250a, irrigation supply line 255a, gas feed line 240b, lens wash feed line 245b, suction feed line 250b, irrigation feed line 255b, or the like) and ports (e.g., proximal channel opening 110, dual valve wells 135, detachable gas/lens wash connection 290, detachable irrigation connection 293, detachable suction connection 295, or the like), and/or cleaning the exterior of the device. After manual cleaning, the endoscope 100 may be visually inspected to verify that the endoscope 100 is clean and free from defects. Next, high-level disinfection (HLD) or sterilization may occur. After HLD or sterilization, the endoscope 100 may be stored for future use.

It is contemplated that prior to storing the endoscope 100, it may be desirable to assess the lumens (e.g., working channel 235, gas supply line 240a, lens wash supply line 245a, suction supply line 250a, irrigation supply line 255a, gas feed line 240b, lens wash feed line 245b, suction feed line 250b, irrigation feed line 255b, or the like) of the endoscope 100 for moisture or foreign matter (e.g., water, isopropyl alcohol, detergent (wet, damp, or dried), organic matter (wet, damp, or dried), and/or any other solid, fluid, or liquid that the endoscope 100 may come into contact with during or after the procedure). For example, having the ability assess the lumens of the endoscope 100 for moisture and/or foreign matter without “fouling” or “contaminating” the endoscope 100 (e.g., without requiring additional reprocessing after the verification) in a quick, easy, and/or cost effective manner may improve the overall hygiene of re-usable endoscopes 100, decrease the risk of hospital acquired infections (HAIs), eliminate (or substantially reduce) the need for labor-intensive borescope inspections, and/or allow facilities to inspect each and every endoscope at the end of processing rather than periodic random sampling.

Typically, the endoscope 100, working channel 235, and/or other portions of the endoscope 100 may be formed from low coefficient of friction polymer materials, such as, but not limited to, polytetrafluoroethylene (PTFE) or other fluorinated polymers. The resistivity of these materials is typically on the order of about 1018 ohms. Said differently these types of materials strongly resist electrical current. Surface resistivity is a property of the material and remains constant regardless of the configuration of the electrodes used for the measurement. Thus, surface resistivity of the endoscope 100 or lumens thereof may be used to identify moisture and/or foreign matter (wet, damp, or dry) within the lumens of the endoscope 100. For example, if moisture or foreign matter is present within the lumen(s) of the endoscope 100, the surface resistivity of the endoscope 100 or portions thereof may decrease as the surface resistivity of water, isopropyl alcohol, detergents, organic matters, or the like is less than the surface resistivity of fluorinated polymers.

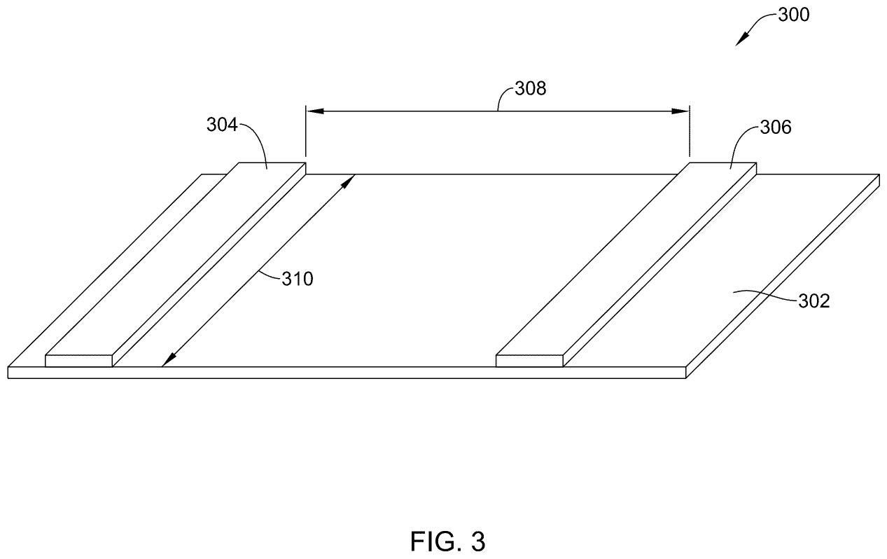

Surface resistivity may be measured using two electrodes in contact with the surface for which resistivity is being measured. FIG. 3 is a schematic diagram of a basic system for measuring surface resistivity of a generic object 302 having a width 310. The system 300 may include the object 302 for which surface resistivity is to be measured, a first electrode 304, and a second electrode 306. The first and second electrodes 304, 306 may be in contact with a surface of the object 302. The first and second electrodes 304, 306 may be spaced from one another by a length or distance 308. In the illustrated setup, the surface resistivity of the object 302 can be calculated using Equation 1:

$${ρ}_{S} = \frac{\frac{U}{L}}{\frac{{I}_{S}}{W}} \qquad Equation 1$$

where ρₛ is the surface resistivity, U is the direct current (DC) voltage drop between the first electrode 304 and the second electrode 306, L is the distance 308 between the first electrode 304 and the second electrode 306, Iₛ is the surface current, and W is the width 310 of the object 302. Said differently, the surface resistivity may be determined by the ratio of the DC voltage drop per unit length to the surface current per unit width.

The surface resistivity of an inner surface of a tubular member may be calculated using Equation 2:

$${ρ}_{S} = \frac{\frac{U}{L}}{\frac{{I}_{S}}{π D}} \qquad Equation 2$$

where ρₛ is the surface resistivity, U is the direct current (DC) voltage drop between the first electrode and the second electrode, L is the distance between the first electrode and the second electrode, Iₛ is the surface current, and D is the inner diameter of the tubular member. Said differently, the surface resistivity may be determined by the ratio of the DC voltage drop per unit length to the surface current per unit width (e.g., the circumference of the lumen).

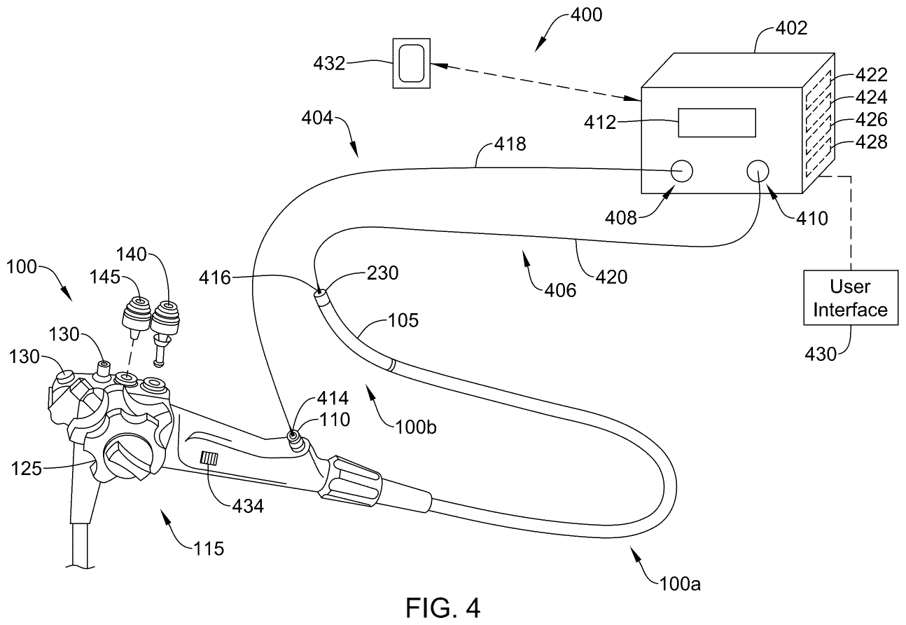

FIG. 4 is a perspective view of an illustrative system 400 for measuring surface resistivity in an endoscope 100. As described above, the illustrative system 400 may be used to measure surface resistivity in devices other than an endoscope. The system 400 may include a power and control element 402 which supplies the necessary electrical energy to a first probe 404 and a second probe 406. The power and control element 402 may be configured to measure surface resistivity within a lumen, such as, but not limited to, the working channel 235 of the endoscope 100. While the system 400 is described with respect to the working channel 235, it is contemplated that the system 400 may be used with other lumens as well. The power and control element 402 may house the DC circuitry 422, a controller or processing module 424 in communication with a memory 426, and a power source 428. The first probe 404 and the second probe 406 may each include an electrical connection 418, 420 configured to be electrically connected to the power and control element 402 adjacent to the proximal ends 408, 410 of the probes 404, 406.

The power and control element 402 may be configured to compare a measured surface resistivity with an expected surface resistivity of a working channel 235 with no moisture, liquid, and/or foreign matter present. In some cases, the power and control element 402 may be configured to receive an input indicating the type of device to be tested and may use information particular to the device to be tested to determine if moisture, liquid, and/or foreign matter is present. In some cases, the power and control element 402 may be in communication with a user interface 430 (e.g., keyboard, mouse, touchscreen display, or the like) configured to receive the user input. Alternatively, or additionally, the power and control element 402 may be configured to wirelessly receive the input from a remote device 432 (e.g., a dedicated input device, a smartphone, a tablet computer, laptop, desktop computer, or the like). Alternatively, or additionally, a user may scan a machine-readable representation of information 434, such as, but not limited to, a barcode, a QR code, an RFID tag, or the like. The machine-readable representation of information 434 may include the device type and/or information related to the surface resistivity. In some cases, the machine-readable representation of information 434 may be secured or otherwise positioned on the endoscope. However, this is not required. The power and control element 402 may include a database of information stored in the memory 426 thereof specific to different devices and/or parts of the device that the processing unit 424 within the power and control element 402 can reference when determining the surface resistivity. Such information may include surface resistivity of the dry and/or contaminant-free material, an inner diameter of the lumen to be tested, a distance between the proximal end of the lumen and the distal end of the lumen (or a distance between electrode connection points), or the like. It is contemplated that other electrical properties, such as, but not limited to, surface impedance, surface conductivity, or the like may be used in addition to or in place of surface resistivity. In such an instance, the memory 426 and/or the machine-readable representation of information 434 may include information related to surface impedance of a dry and/or contaminant-free device, surface conductivity of a dry device, information required to calculate surface impedance and/or surface conductivity, or the like.

The power and control element 402 may include a display 412 configured to display an output. In some cases, the display 412 may be configured to display a variable output such as, but not limited to, a numeric value or graduated scale of the surface resistivity. In other embodiments, the display 412 may be configured to display a binary indication of the presence of moisture, liquid, foreign matter, or the like in the working channel 235. For example, the display 412 may be configured to display a green light if the measured surface resistivity indicates there is no moisture, liquid, foreign matter, or other contaminant present in the working channel 235 or a red light if the measured surface resistivity indicates there is moisture, liquid, foreign matter, or other contaminant present in the working channel 235. This is just an example. Other visual, audio, and/or haptic indicators may be used, as desired. In some cases, the power and control element 402 may include signal amplification if the resistivity of the material of the channel is very low.

The first probe 404 may include a first electrode 414 affixed and electrically coupled to the distal end of the electrical connection 418. The first electrode 414 may be electrically coupled to the power and control element 402 via the electrical connection 418. Similarly, the second probe 406 may include a second electrode 416 affixed and electrically coupled to the distal end of the electrical connection 420. The second electrode 416 may be electrically coupled to the power and control element 402 via the electrical connection 420. The first and second electrodes 414, 416 may be configured to be inserted into the working channel 235 or otherwise contact an inner surface of the working channel 235. In some cases, the first electrode 414 may be in contact with an inner surface of the working channel 235 adjacent to the proximal channel opening 110 and the second electrode 416 may in contact with an inner surface of the working channel 235 adjacent to the distal opening 230 thereof. It is contemplated that the first and/or second electrodes 414, 416 may be positioned at other locations along a length of the working channel 235, as desired.

In some embodiments, the first and/or second probes 404, 406 and the components thereof may be single use devices and may be disposed of after each testing procedure. For example, the first and/or second probes 404, 406 may be provided in a disinfected or sanitized state and individually packaged such that touching the reprocessed endoscope 100 with the first and/or second probes 404, 406 does not introduce contaminants to the endoscope 100. In other examples, the first and/or second probes 404, 406 may be reusable. For example, the first and/or second probes 404, 406 may be placed in an alcohol bath between uses. The first and/or second probes 404, 406 may be dried prior to positioning the first and/or second electrodes 414, 416 in contact with the endoscope 100 to avoid introducing moisture into the device.

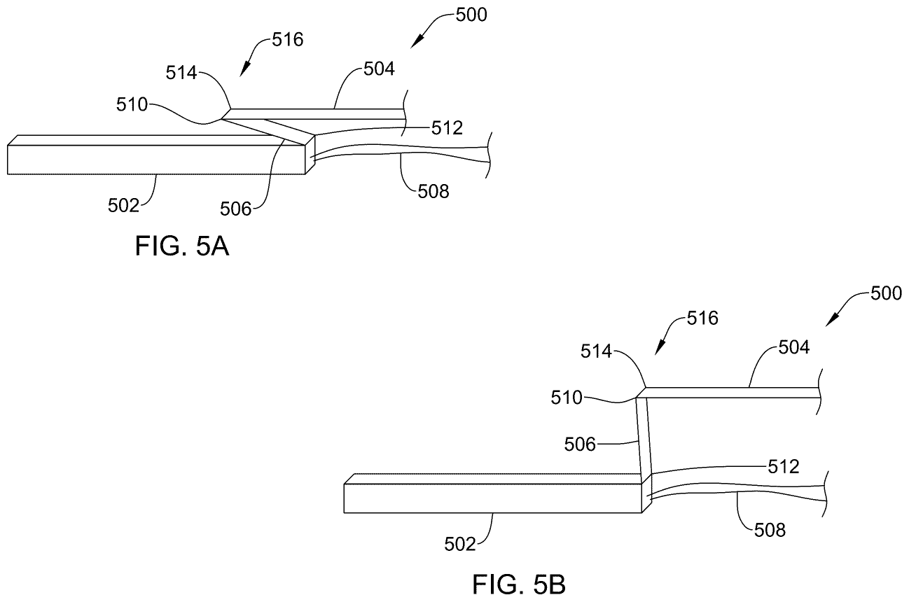

FIG. 5A is a perspective view of a distal end region of an illustrative probe 500 in a first configuration and FIG. 5B is a perspective view of the distal end region of the illustrative probe 500 in a second configuration. The probe 500 may include an electrode 502, an actuation member 504, a biasing member 506, and an electrical connection 508. The electrical connection 508 may be electrically coupled to the electrode 502 and may extend proximally to a power and control element, such as, but not limited to, the power and control element 402 described herein. Generally, the probe 500 include an electrode assembly 516 that is actuatable or movable between a first radially collapsed insertion configuration, as shown in FIG. 5A and a second radially expanded use configuration, as shown in FIG. 5B. For example, the electrode assembly 516 may be introduced into the working channel 235 in the radially collapsed configuration. The actuation member 504 may then be proximally retracted to move the electrode 502 radially outwards to bring the electrode 502 into contact with the inner surface of the working channel 235 or lumen. It is contemplated that the biasing member 506 may be movable between an orientation in which a longitudinal axis of the biasing member 506 is parallel to or substantially parallel to a longitudinal axis of the electrode 502 and an orientation in which the longitudinal axis of the biasing member 506 is generally orthogonal to the longitudinal axis of the electrode 502. In some cases, the longitudinal axis of the biasing member 506 may extend at a non-parallel or non-orthogonal angle to the longitudinal axis of the electrode 502 to allow the electrode assembly 516 to be used in lumens of varying inner diameters. The biasing member 506 may extend from a proximal end 510 pivotably coupled to the actuation member 504 to a distal end 512 pivotably coupled to the electrode 502. The biasing member 506 may be configured to push the electrode 502 radially outwards as the actuation member 504 is proximally retracted. It is contemplated that the biasing member 506 may take any number of shapes and/or configurations. For example, the biasing member 506 may be a spring, a rod, an elongate planar shape, such as, but not limited to a rectangular prism, or the like.

The biasing member 506 can be made from a number of different materials such as, but not limited to, metals, metal alloys, shape memory alloys and/or polymers, as desired, enabling the electrode 502 to be biased radially outward. Depending on the material selected for construction, the biasing member 506 may be self-expanding (i.e., configured to automatically expand when unconstrained). As used herein the term “self-expanding” refers to the tendency of the biasing member 506 to return to a preprogrammed shape when unrestrained from an external biasing force. The biasing member 506 may be electrically insulated from the electrode 502.

The actuation member 504 may extend proximally from a distal end 514 coupled to the biasing member 506. The actuation member 504 may be a pull-wire, an elongate filament, an elongate rod, an elongate bar, or the like. The actuation member 504 may be electrically insulated from the electrode 502. The actuation member 504 may have a length which allows the proximal end (not explicitly shown) of the actuation member 504 to extend proximally from the working channel 235 or lumen and to be gripped by the user. It is contemplated that the user may proximally actuate the actuation member 504 to move the electrode 502 radially outwards and distally actuation the actuation member 504 to move the electrode 502 radially inwards. The reverse configuration is also contemplated in which distal actuation of the actuation member 504 moves the electrode 502 radially outwards and proximal actuation of the actuation member 504 moves the electrode 502 radially inwards. In some embodiments, the user may grip the electrical connection 508 to maintain the electrode 502 in a desired position while the actuation member 504 is actuated. In other examples, a separate structure may be provided to maintain the electrode 502 in a desired position while the actuation member 504 is actuated

The electrode 502 may take any number of shapes desired. For example, the electrode 502 may be a rectangular prism, as shown. In other examples, the electrode 502 may have a generally tubular shape. It is contemplated a tubular electrode may include a plurality of intersecting struts defining a plurality of recesses similar to a stent. Such a structure may be radially expandable and/or collapsible by attaching the actuation member 504 directly to the tubular electrode. For example, a tubular electrode may be elongated or lengthened to reduce a diameter of the tubular electrode or contracted or reduced in length to expand a diameter of the tubular electrode. The electrode 502 need not extend around an entirety of the inner diameter of the working channel 235 or lumen. The electrode 502 may take other shapes, such as, but not limited to, clamps, clips, cylindrical, rod-like, concave, or the like.

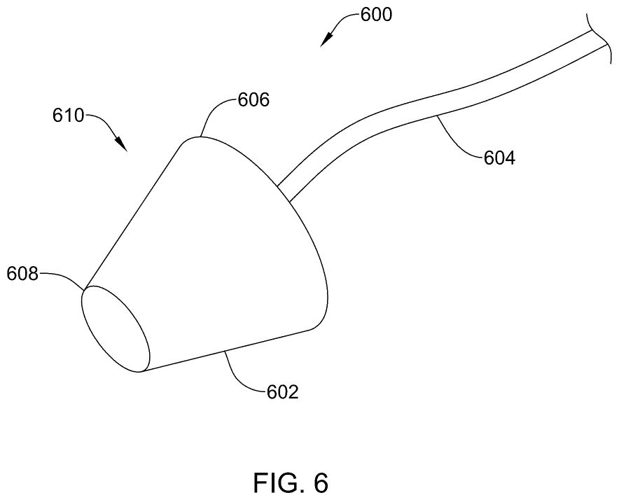

FIG. 6 is a perspective view of a distal end region of another illustrative probe 600. The probe 600 may include an electrode 602 and an electrical connection 604. The electrical connection 604 may be electrically coupled to the electrode 502 and may extend proximally to a power and control element, such as, but not limited to, the power and control element 402 described herein. The electrode 602 may include a body portion 610 extending from a proximal end 606 to a distal end 608. The body portion 610 may be generally solid. However, this is not required. In some cases, a lumen or cavity may extend through a length or partially through a length of the body portion 610, as desired.

In some embodiments, the body portion 610 may have an outer diameter which tapers or reduces from the proximal end 606 to the distal end 608 thereof. It is contemplated that a tapered body portion 610 may allow the electrode 602 to form a friction fit with lumens having different sized inner diameters. In some examples, the body portion 610 may include regions having a generally constant outer diameter. These regions of generally constant outer diameter may include sloped transitions or abrupt, stair-step transitions therebetween. The body portion 610 may take any shape desired, such as, but not limited to, a truncated cone, a cone, a rectangular prism, a sphere, a hemi-sphere, an ovoid, polygonal, a helically wound coil, irregular, or the like.

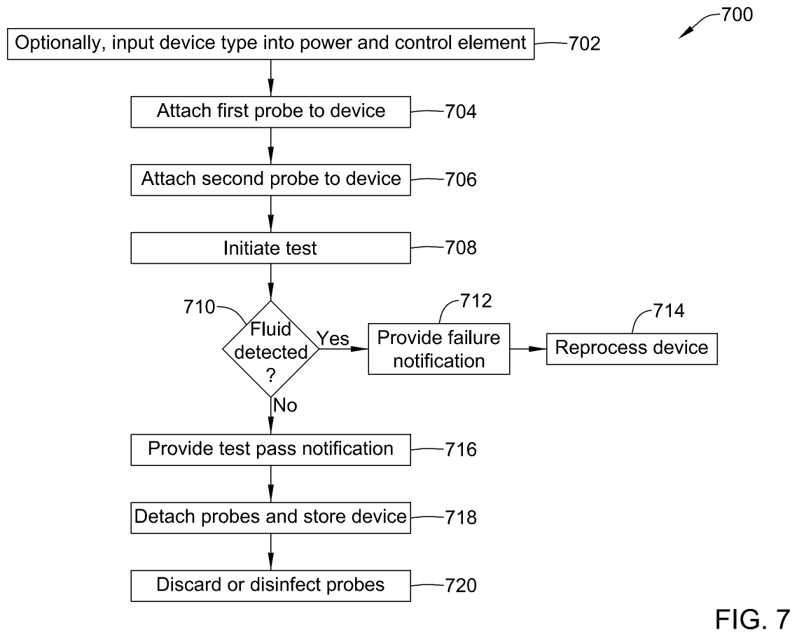

FIG. 7 is a schematic flow chart of an illustrative method for using the system 400 for measuring surface resistivity of a device to detect moisture and/or foreign matter within a lumen of the device. The illustrative method 700 may be performed on each and every device that undergoes reprocessing (if desired) without requiring additional reprocessing when the device passes, or moisture or foreign matter is not detected. To begin, the type and/or model of the device to be tested may be optionally input into the power and control element 402, as shown at block 702. This may include receiving a user input at a user interface 430 coupled to or formed with the power and control element 402, receiving the input at a remote device 432 and wirelessly transmitting the information to the power and control element 402, scanning a machine-readable representation of information 434 on the device, or the like. Next, the first probe 404 may be attached to the device to be tested, such as, but not limited to, an endoscope 100, as shown at block 704. The first probe 404 may be coupled to the endoscope 100 such that the first electrode 414 is in mechanical and/or electrical contact with the surface to be tested. In one illustrative example, the first probe 404 may be coupled to the endoscope 100 such that the first electrode 414 is in contact with the inner surface of the proximal channel opening 110 of the working channel 235. This is just one example. The first electrode 414 may be positioned in contact with an inner surface of the working channel 235 adjacent to the distal opening 230 thereof. In other examples, the first probe 404 and the first electrode 414 may be coupled to an end of a different lumen of the endoscope 100. Next, the second probe 406 may be attached to the device to be tested, such as, but not limited to, an endoscope 100, as shown at block 706. The second probe 406 may be coupled to the endoscope 100 such that the second electrode 416 is in mechanical and/or electrical contact with the surface to be tested. It is contemplated that the first and second probes 404, 406 may be coupled to opposing ends of same lumen. In one illustrative example, the second probe 406 may be coupled to the endoscope 100 such that the second electrode 416 is in contact with the inner surface of the distal opening 230 of the working channel 235. This is just one example. The second electrode 416 may be positioned in contact with an inner surface of the working channel 235 adjacent to the proximal channel opening 110 thereof. In other examples, the second probe 406 and the second electrode 416 may be coupled to an end of a different lumen of the endoscope 100. It is contemplated that in some cases, the second probe 406 may be coupled to the device prior to coupling the first probe 404. It is further contemplated that the first and/or second probes 404, 406 may be coupled to the device to be tested prior to input the device type and/or model into the power and control element 402.

After the first and second probes 404, 406 are secured to the device such that the first and second electrodes 414, 416 are spaced from one another and in contact with a surface of a same lumen, the surface resistivity test may be initiated, as shown at block 708. The test may be initiated through actuation of a button on the power and control using 402, actuation of a button on a touch screen interface (such as, but not limited to, the user interface 430), receiving an input from a remote device 432, or the like. Upon initiation of the test, the power and control element 402 may transmit an electrical signal through the electrical connectors 418, 420 to determine a DC voltage drop between the first and second electrodes 414, 416. The processing unit 424 may use the DC voltage drop, the inner circumference of the lumen (e.g., inner circumference of the working channel 235 in the current example), and a distance between the electrodes 414, 416 to calculate a surface resistivity of the working channel 235 of the endoscope 100. The processing unit may then determine if the calculated surface resistivity of the working channel 235 is within a predetermined threshold of the surface resistivity of a dry or uncontaminated (e.g., free from organic matter, detergents, water, isopropyl alcohol, or other contaminants) working channel 235 to determine if moisture, liquid, and/or foreign matter is present, as shown at block 710. For example, if the calculated surface resistivity is below a predetermined threshold or value, the processing unit 424 may determine there is moisture, liquid, and/or foreign matter in the working channel as the moisture, liquid, foreign matter, or the like is more conductive than the material forming the working channel 235 and thus lowers surface resistivity. In some cases, the predetermined threshold or value may be the surface resistivity of the dry working channel 235. In other embodiments, the predetermined threshold or value may be the surface resistivity of the dry working channel 235 plus or minus a predetermined offset. If the processing unit 424 determines that there is liquid or other contaminant in the working channel 235, the processing unit 424 may provide a test failure notification, as shown at block 712. The test failure notification may be displayed on the display 412 of the power and control element 402 (or at a remote device, such as, but not limited to, remote device 432) as a numerical value, a binary alert, an audio alert, a haptic alert, or the like. If the endoscope 100 fails the moisture and/or foreign matter test, the first and/or second probes 404, 406 may be detached from the endoscope 100 and the endoscope 100 may be reprocessed, as shown at block 714.

If the processing unit 424 determines that there is no liquid or other contaminant in the working channel 235, the processing unit 424 may provide a test pass notification, as shown at block 716. The test pass notification may be displayed on the display 412 of the power and control element 402 (or at a remote device, such as, but not limited to, remote device 432) as a numerical value, a binary alert, an audio alert, a haptic alert, or the like. When the endoscope 100 passes the moisture and/or foreign matter test, the first and/or second probes 404, 406 may be detached from the endoscope 100 and the endoscope 100 may be prepared for storage and stored until its next use, as shown at block 718. The first and/or second probes 404, 406 may be discarded if they are single use or may be disinfected and stored if they are reusable.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A method for detecting a presence of moisture and/or foreign matter in a lumen of a medical device, the method comprising:
coupling a first probe to a first end of a lumen of a medical device, the first probe comprising a first electrode and a first electrical connection extending proximally from the first electrode;
coupling a second probe to a second end of the lumen of the medical device, the second probe comprising a second electrode and a second electrical connection extending proximally from the second electrode;
transmitting an electrical signal through the first electrical connection and the second electrical connection;
determining a direct current (DC) voltage drop between the first electrical connection and the second electrical connection;
calculating a surface resistivity of the lumen based on the DC voltage drop;
determining if the calculated surface resistivity is below a threshold surface resistivity; and
providing a test status notification.

2. The method for detecting a presence of moisture and/or foreign matter in a lumen of a medical device of claim 1, further comprising inputting a device type or model into a control and power element.

3. The method for detecting the presence of moisture and/or foreign matter in a lumen of a medical device of claim 2, wherein the control and power element is configured to use characteristics of the device type or model to calculate the surface resistivity of the lumen.

4. The method for detecting a presence of moisture and/or foreign matter in a lumen of a medical device of claim 3, wherein the characteristics of the device type or model comprise an inner diameter of the lumen and a distance between the first electrode and the second electrode.

5. The method for detecting a presence of moisture and/or foreign matter in a lumen of a medical device claim 1, wherein coupling the first probe to the first end of the lumen of the medical device comprises positioning the first electrode in contact with an inner surface of the lumen.

6. The method for detecting a presence of moisture and/or foreign matter in a lumen of a medical device of claim 1, wherein coupling the second probe to the second end of the lumen of the medical device comprises positioning the second electrode in contact with an inner surface of the lumen.

7. The method for detecting a presence of moisture and/or foreign matter in a lumen of a medical device of claim 1, wherein providing the test status notification comprises displaying a numerical representation of the calculated surface resistivity.

8. The method for detecting a presence of moisture and/or foreign matter in a lumen of a medical device of claim 1, wherein providing the test status notification comprises displaying a binary representation of the presence of moisture and/or foreign matter in the lumen.

9. The method for detecting a presence of moisture and/or foreign matter in a lumen of a medical device of claim 1, wherein providing the test status notification comprises providing a test pass notification indicating the lumen is free from moisture and/or foreign matter or a test failure notification indicating moisture and/or foreign matter is present in the lumen.

10. A method for detecting a presence of moisture and/or foreign matter in a lumen of a medical device, the method comprising:
receiving at a power and control element a device type and/or model of a medical device;
positioning a first electrode in contact with a first end of a lumen of the medical device, the first electrode in electrical communication with the power and control element;
positioning a second electrode in contact with a second end of the lumen of the medical device, the second electrode in electrical communication with the power and control element;
determining a direct current (DC) voltage drop between the first electrode and the second electrode;
calculating a surface resistivity of the lumen based at least in part on the DC voltage drop; and
determining if the calculated surface resistivity is below a threshold surface resistivity;
wherein if the calculated surface resistivity is below the threshold surface resistivity displaying a test failure notification and if the calculated surface resistivity is not below the threshold surface resistivity displaying a test pass notification.

11. The method for detecting a presence of moisture and/or foreign matter in a lumen of a medical device of claim 10, wherein receiving at the power and control element the device type and/or model of the medical device comprises receiving a user input at the power and control element.

12. The method for detecting a presence of moisture and/or foreign matter in a lumen of a medical device of claim 10, wherein receiving at the power and control element the device type and/or model of the medical device comprises scanning a machine-readable representation of information.

13. The method for detecting a presence of moisture and/or foreign matter in a lumen of a medical device of claim 10, wherein the test failure notification and/or the test pass notification comprise a visual alert.

14. The method for detecting a presence of moisture and/or foreign matter in a lumen of a medical device of claim 10, wherein the test failure notification and/or the test pass notification comprise an audio alert.

15. A system for detecting moisture and/or foreign matter in a lumen of a medical device, the system comprising:
a first probe comprising a first electrode and a first electrical connection extending proximally from the first electrode;
a second probe comprising a second electrode and a second electrical connection extending proximally from the second electrode; and
a power and control element including a processing unit and a power source, the power and control element electrically coupled to a proximal end of the first electrical connection and a proximal end of the second electrical connection;
wherein when the first electrode is electrically coupled to an inner surface of a lumen and the second electrode is electrically coupled to the inner surface of the lumen and spaced a distance from the first electrode, the power and control element is configured to calculate a surface resistivity of the lumen.

16. The system for detecting moisture and/or foreign matter in a lumen of a medical device of claim 15, wherein the first electrode is coupled to an actuation member.

17. The system for detecting moisture and/or foreign matter in a lumen of a medical device of claim 16, wherein actuation of the actuation member is configured to radially displace the first electrode.

18. The system for detecting moisture and/or foreign matter in a lumen of a medical device of claim 16, further comprising a biasing member coupled to the first electrode and the actuation member.

19. The system for detecting moisture and/or foreign matter in a lumen of a medical device of claim 15, wherein the first electrode comprises a body portion having a tapered outer diameter.

20. The system for detecting moisture and/or foreign matter in a lumen of a medical device of claim 15, wherein the power and control element further comprises a display, the display configured to provide a positive or negative indication of the presence of moisture and/or foreign matter in the lumen.
